(19) United States
(12) Patent Application Publication
(10) Pub. No.: US 2006/0199185 A1
(43) Pub. Date: Sep. 7, 2006

(54) Plasmid having a function of t-vector and expression vector, and expression of the target gene using the same

(76) Inventors: Young Shin Song, Daejeon (KR); Haryoung Poo, Daejeon (KR); Moon-Hee Sung, Daejeon (KR); Seung-Pyo Hong, Daejeon (KR); Yoon Ho Choi, Daejeon (KR); Kwang Kim, Daejeon (KR); Il Han Lee, Gyeonggi-do (KR); Je Hyun Park, Daejeon (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/564,880

(22) PCT Filed: Dec. 31, 2003
(86) PCT No.: PCT/KR03/02927

§ 371 Date: Jan. 17, 2006

(30) Foreign Application Priority Data
Jul. 16, 2003 (KR) 10-2003-0048625

(51) Int. Cl. C40B 40/02; C40B 40/08; C12Q 1/68; C12N 15/74

(52) U.S. Cl. 435/6; 435/69.1; 435/252.3; 435/471

(57) ABSTRACT

The present invention relates to a plasmid (pHCE-FOREX) functioning as both a T-vector and an expression vector, which is produced by imparting a T-vector function to an HCE promoter derived from a constitutive high-level expression vector and can express a target protein in a simple and rapid manner. Also, the present invention relates to an expression vector having the target gene inserted into the plasmid, and the expression of the target gene using the same. The plasmid of the present invention can be converted into a vector that expresses the target protein by one-step T-vector cloning in a simple and rapid manner. The plasmid converted into the expression vector does not require a re-transformation step and allows the high-level expression of the target protein only by the culturing of transformed E. coli without the addition of an expensive inducer. Thus, according to the present invention, expression plasmids for large amounts of target genes can be produced at the same time, so that the present invention will be very efficient in establishing expression systems for certain genomes and gene groups.

### TECHNICAL FIELD

The present invention relates to a plasmid functioning as both a T-vector and an expression vector. Moreover, the present invention relates to an expression vector having a target gene inserted into the plasmid, and the expression of the target gene using the same.

### BACKGROUND ART

A typical method of expressing a target gene using a vector having the target gene inserted therein includes a method in which the target gene is amplified by polymerase chain reaction (PCR) and then inserted into the expression vector. The gene amplification product resulting from this PCR has one additional nucleotide having a adenine base at the 3′-terminal end, due to the terminal transferase activity of a Taq DNA polymerase used in the PCR reaction (Clark, J. M., Nucleic Acid Res., 16:9677, 1988).

Owing to the specific characteristic of the gene amplification product, the gene amplification product should be subjected to a process of making its end blunt or cohesive by restriction enzyme or terminal transferase treatment, before it is cloned into a plasmid vector. Thus, there are problems in that the gene cloning requires several steps, has reduced efficiency and is difficult to perform.

In order to overcome such problems and to easily clone the PCR amplification gene product, a T-vector was developed, which is a linear vector containing one additional nucleotide having thymine bases at both 3′-terminal ends.

As a method of producing the T-vector, there is a method of artificially adding a nucleotide having thymine base at the 3′-end. Namely, the liner T-vector containing an additional nucleotide having a thymine base at the 3′-terminal end can be constructed by a method where a cloning vector is cut with a restriction enzyme capable of making its end blunt, and the linear vector having the blunt end is added either with deoxythymidine triphosphate (dTTP) by means of a Taq DNA polymerase (Marchunk, D. et al., Nucleic Acid Res., 19:1154, 1991), or with dideoxythymidine triphosphate (ddTTP) by means of terminal deoxynucleotidyl transferase (Holton, T. A. et al., Nucleic Acid Res., 19:1156, 1991).

However, in this method, an incomplete T-vector having no additional thymidine nucleotide can be produced due to non-optimal reaction conditions or inactivated enzymes, since the T-vector is produced in a manner dependent on the activity efficiency of the terminal transferase. For this reason, this method has a problem in that the frequency of a self-ligated product that was not cloned in a cloning process is increased. Also, there is a problem in that a host cell is transformed with this product so that the cloning efficiency of the amplified gene product is reduced.

As a second method of producing the T-vector, there is a method of using restriction enzyme AspEI (Yoshikazu, I. et al., Gene, 130:152, 1993), HphI (David, A. M. et al., Bio/Technology, 9:65, 1991), MboII or XcmI, which can leave only one nucleotide having a thymine base at the 3′-terminal end, when digesting the gene with the restriction enzyme.

In this method, an oligonucleotide is synthesized which was designed so that, when two restriction enzyme recognition sites to be used were arranged in parallel and the gene was cut with the restriction enzyme, only one thymidine nucleotide remains at the 3′-end of the cut vector. The synthesized oligonucleotide is inserted into a parent vector, and cut with the restriction enzyme, to produce a T-vector. However, this method has a problem in that it cannot be used when the restriction enzyme recognition sites are present in the parent vector.

An analysis based on pUC19 that is frequently used as the parent vector shows that HphI and MboII recognition sites are present in seven places, respectively, an AspEI recognition site is present in one place and only XcmI recognition site is not present. Thus, many studies on the development of the T-vector using XcmI are being conducted (Kovalic, D. et al., Nucleic Acid Res., 19:4560, 1991; Cha, J. et al., Gene, 136:369, 1993; Testoris, A. et al., Gene, 143:151, 1994; Harrison, J. et al., Anal. Biocdem., 216:235, 1994; and Boroskov, A. Y. et al., Biotechniques, 22:812, 1997).

Even in this case, however, there is a problem in that the difference in the distance of migration between an incompletely cut vector and a completely cut vector on agarose electrophoresis gel is too small, since a gene fragment, which is released when digesting the oligonucleotide with the restriction enzymes to produce the T-vector, is too small. Thus, even when only the completely cut T-vector is separated by a gel extraction method, the incompletely cut vector will also be present. As a result, since a portion of the vector, which was incompletely cut with the restriction enzymes, is used in cloning the amplified gene produce as it is, the self-ligated vector is found in transformed E. coli at high ratio so that the cloning efficiency of the gene amplification product is reduced.

To overcome this problem, there have been continued efforts to develop a technology for producing a T-vector by which a gene fragment that was cut and separated on agarose gel electrophoresis can be clearly distinguished, the problem in that the incomplete vector is produced and thus only the vector where the amplified gene product was not cloned due to the self-ligation is transformed, can be solved, so that the ratio of the self-ligated vector found on transformation can be reduced, thereby increasing the cloning efficiency of the amplified gene product.

Meanwhile, in general studies on the expression of large amounts of a target protein, a step of establishing an expression plasmid suitable for an expression system is first performed. In establishing the expression plasmid, the production of an oligonucleotide, which is used in amplifying a target protein gene to be inserted into a vector, comprises analyzing the base sequence of a gene encoding a target protein, and inserting restriction enzyme recognition sites, which are not present in the base sequence of the target gene. This facilitates the cloning of the amplified product into the expression vector.

As a result, the produced oligonucleotide contains an extra oligonucleotide for adding the restriction enzyme recognition sites, in addition to the base sequence of the target gene, which is used as a template. For this reason, in amplifying the target gene by PCR using this oligonucleotide, there is a problem in that the efficiency which the oligonucleotide is annealed specifically to the target gene is lowered so that it is difficult to selectively amplify only the target gene. Furthermore, since the restriction enzyme recognition sites, which are inserted into the oligonucleotide used for gene amplification to facilitate the cloning, are located at both ends of the amplified target gene product, there is the problem of low digesting efficiency.

Thus, to increase the efficiency of a plasmid for the expression of a target protein, a method is proposed which comprises the steps of: cloning an amplified target gene product into a T-vector; selecting a T-vector clone containing the target gene; digesting the T-vector with restriction enzymes; and establishing a final plasmid using the cut T-vector. However, this method is inconvenient in that it requires a two-step process.

In the case of expressing a target protein at large amounts, most of its purpose is the industrial production of the target protein in a more simple, efficient and economic manner, and thus, a need for the development of a gene expression system having excellent industrial usefulness, such as more stable gene expression and cost saving, is continuously raised, and efforts for satisfying this need is continually performed.

Accordingly, the present inventors have conducted intensive studies in an attempt to establish a final expression vector allowing the high-level expression of target proteins, only by simple T-vector cloning to express a target protein, and consequently, found that even when the target protein gene amplified by PCR is cloned into a vector for constitutive high-level expression by one-step cloning, the high-level expression of the target protein gene would be possible, and also this vector could be very efficiently used in the expression of large amounts of target genes, such as the establishment of a whole expression system for microbial genomes, thereby perfecting the present invention.

### DISCLOSURE OF INVENTION

An object of the present invention is to provide a plasmid which functions as both a T-vector and an expression vector and is useful for the construction of a vector that expresses the gene of a target protein in a simple and rapid manner, and also to provide a producing method thereof.

Another object of the present invention is to provide an expression vector having a target gene inserted into the plasmid, and also microorganisms transformed with the expression vector.

Still another object of the present invention is to provide a method for expressing the target gene, which comprises culturing the transformed microorganisms.

Further another object of the present invention is to provide a vector library system, which expresses a large amount of target genes at the same time in an efficient and economic manner.

To achieve the above objects, the present invention provides a plasmid wherein two restriction enzyme recognition sites into which a T-vector can be cloned are introduced at the downstream of a promoter of a vector that is constantly expressed at high levels regardless of the kind of a host cell, whereby the plasmid functions as both the T-vector and an expression vector and has the property of allowing the expression of a target gene to be examined only by one-step T-vector cloning.

Preferably, the restriction enzyme recognition sites into which the T-vector can be cloned are selected from the group consisting of HphI, MboII, AspEI and XcmI, and a polynucleotide is inserted between the two restriction enzyme recognition sites of the plasmid.

When the plasmid according to the present invention is cut with the restriction enzymes, a nucleotide having thymine bases at both 3′-ends of the removal position of the inserted polynucleotide is exposed to function as the T-vector.

In a preferred embodiment, the constitutive high-level expression vector is pHCE, and the present invention provides a plasmid (pHCE-FOREX) functioning as both a T-vector and an expression vector, wherein two AspEI restriction enzyme recognition sites are introduced at the downstream of the HCE promoter of the pHCE vector, and a polynucleotide having AspEI restriction enzyme recognition sites at its both ends is inserted between the two AspEI restriction enzyme recognition sites.

Moreover, the present invention provides a constitutive high-level expression vector (pHCE-FOREX-T), which is obtained by digesting the plasmid pHCE-FOREX with an AspEI restriction enzyme, to remove the polynucleotide having AspEI restriction enzyme recognition sites at its both ends, and in which a nucleotide having thymine bases at both 3′-ends of the removal position of the polynucleotide is exposed.

Furthermore, the present invention provides a method for producing a plasmid (pHCE-FOREX) functioning as both a T-vector and an expression vector, the method comprising the steps of: (a) constructing pHCE-M1 which AspEI restriction enzyme recognition sites were removed by inducing point mutation in AspEI restriction enzyme recognition sites in a pHCE vector; (b) constructing the pHCE-M2 by introducing two AspEI restriction enzyme recognition sites into the downstream of the HCE promoter of the pHCE-M1 vector by PCR using primers containing the AspEI restriction enzyme recognition sites; and (c) inserting a polynucleotide having AspEI restriction enzyme recognition sites at its both ends, between the two AspEI restriction enzyme recognition sites of the pHCE-M2 vector.

Also, the present invention provides an expression vector, which is obtained by digesting the plasmid with the restriction enzymes to remove the inserted polynucleotide, and then inserting a gene encoding a target protein, into a position from which the polynucleotide was removed.

Also, the present invention provides an expression vector wherein a gene encoding a target protein is inserted into the constitutive high-level expression T-vector (pHCE-FOREX-T).

The target protein-encoding gene is preferably a gene amplified by PCR. Also, the gene is preferably a PCR product amplified by using a primer having the amino terminal end of ATG, and a primer specific to the base sequence of the gene, and an NdeI restriction enzyme recognition site is preferably formed in the gene insertion position.

Furthermore, the present invention provides microorganisms transformed with the expression vector, and also a method for expressing a target protein, which comprises culturing the transformed microorganisms.

Moreover, the present invention provides an expression vector library, wherein the library of various genes is inserted into the plasmid, and also provides an expression vector library wherein the library of various genes is inserted into the high-level expression T-vector (pHCE-FOREX-T).

In addition, the present invention provides a method for determining the cloning of a target gene, the method comprising the steps of: (a) transforming microorganisms with the expression vector library; and (b) culturing the transformed microorganisms.

The inventive method for determining the target gene cloning preferably additionally comprises the steps of: separating a plasmid after the step (b); and digesting the plasmid with an NdeI restriction enzyme.

Hereinafter, the producing method of the high-level expression T-vector (pHCE-FOREX-T) according to the present invention, and the expression method using the T-vector, will be described in detail according to each step.

Step 1: Production of pHCE-M1 Containing Two AspEI Restriction Enzyme Recognition Sites

In order to produce a T-vector using, as a basic framework, a constitutive high-level expression vector (pHCE DNA vector; FERM P-17814) having a high-level constitutive promoter and a multicloning site useful for subcloning, point mutation was induced in AspEI restriction enzyme recognition sites present in the pHCE vector, to produce pHCE-M1 which the restriction enzyme recognition sites were removed.

By PCR using a primer containing two AspEI restriction enzyme recognition sites, pHCE-M2 where the two AspEI restriction enzyme recognition sites have been introduced at the downstream of the HCE promoter of the pHCE-M1 was produced.

Step 2: Production of Plasmid (pHCE-FOREX) for Constitutive High-Level Expression T-Vectors

By PCR using a primer containing AspEI restriction enzyme recognition sites, about 800-bp polynucleotide having AspEI restriction enzymes at its both ends was obtained. It was inserted into the AspEI restriction enzyme recognition sites of the pHCE-M2, to produce a plasmid (pHCE-FOREX) for constitutive high-level T-vectors. This facilitates the separation of a DNA fragment in the subsequent conversion into a T-vector.

Step 3: Conversion of Plasmid (pHCE-FOREX) for Constitutive High-Level Expression Vectors into Constitutive High-Level Expression T-Vector (pHCE-FOREX-T)

The plasmid was separated from the E. coli transformed with the plasmid (pHCE-FOREX) for constitutive high-level expression T-vectors, and cut with an AspEI restriction enzyme. The cut plasmid was developed on agarose gel by electrophoresis, thereby obtaining a constitutive high-level expression T-vector (pHCE-FOREX-T) of about 3,000-bp containing a nucleotide having thymine bases at both 3′-ends of the gene remaining after the separation of about 800-bp polynucleotide.

Step 4: Cloning and High-Level Expression of a Gene Encoding a Target Protein

A gene encoding a target protein was amplified by PCR, and then cloned into the constitutive high-level expression T-vector (pHCE-FOREX-T). The high-level expression T-vector having the target gene inserted therein was designed so that if the start codon of an amino-terminal primer for amplifying a gene encoding a target protein is made of ATG, when T-vector cloning and then forward insertion are performed, an NdeI restriction enzyme recognition site is produced such that it can be easily examined whether the cloning is successful.

If the gene to be expressed in the forward direction is inserted as described above, the over-expression of a target protein can be confirmed without treatment with an expression inducer, at a given time after culturing the transformed E. coli with this plasmid.

The plasmid for high-level expression T-vectors of the present invention can be easily converted into a high-level expression T-vector by separating the plasmid from E. coli transformed with the plasmid for T-vector, digesting the separated plasmid with an AspEI restriction enzyme, and then separating and purifying the rest portion of the plasmid excluding a polynucleotide portion of about 800 bp. Also, the plasmid has an excellent storage property such that it can be stored in a form transformed into E. coli. Furthermore, it allows the examination of expression even when a gene encoding a target protein to be expressed is cloned only by one step. This indicates that the plasmid also has an advantage as a system that can express a target protein regardless of the kind of a host cell.

Furthermore, in establishing a system expressing large amounts of target genes at the same time, the plasmid system according to the present invention shows a far superior efficiency to other existing systems. This is an advantage that can be possessed only by the inventive plasmid system in which the expression plasmid produced by T-vector cloning utilizes a constitutive high-level expression promoter, and can be expressed regardless of the kind of a host cell, and thus allows the immediate examination of expression in a transformant obtained by one-step cloning. Owing to this advantage, the efficiency of establishment of a microbial genome expression system can be significantly improved.

### BRIEF DESCRIPTION OF DRAWINGS

FIG. 1 is an agarose gel electrophoresis photograph for a DNA fragment obtained by digesting a plasmid (pHCE-FOREX) for high-level expression T-vectors with AspEI.

FIG. 2 is a schematic diagram showing a novel high-level expression T-vector (pHCE-FOREX-T) according to the present invention.

FIG. 3 is an agarose gel electrophoresis photograph for hTNF-α amplified by PCR.

FIG. 4 is an agarose gel electrophoresis for DNA fragments obtained by digesting 12 colonies with an NdeI restriction enzyme, in which the colonies were randomly selected for the examination of the cloning using a high-level expression T-vector.

FIG. 5 is an SDS-polyacrylamide gel electrophoresis (SDS-PAGE) photograph for proteins obtained from 12 transformants selected for the examination of cloning.

### DETAILED DESCRIPTION OF THE INVENTION

The present invention will hereinafter be described in further detail by Examples. It will however be obvious to a person skilled in the art that these Examples are given for illustrative purpose only, and the scope of the present invention is not limited to or by these Examples.

In the following Examples, although pHCE was particularly used as a constitutive expression vector, any vector may be used without limitation if it is a vector that is expressed regardless of the kind of a host cell.

Also, in the following Examples, although two AspEI recognition sites were introduced at the downstream of the HCE promoter of pHCE, it will be obvious to a person skilled in the art that in the case of the vector being cut with a restriction enzyme, such as HphI, MboII or XcmI, introducing the recognition site of a restriction enzymes, such as HphI, MboII or XcmI having nucleotide with thymine bases at both 3′-ends exposed, can provide the same result. However, if the restriction enzyme recognition site is present in the constitutive high-level expression vector, such a site should be removed by mutation.

### Example 1

### Production of Plasmid for Constitutive High-Level Expression T-Vectors

In order to remove an AspEI restriction enzyme recognition site present in the existing pCHE vector, point mutation was induced in the AspEI restriction enzyme recognition site by PCR using the primer of SEQ ID NO: 1 below, thereby producing pHCE-M1 which the restriction enzyme recognition site have been removed.

| | |
|---|---|
| 5′-GCCTGGCTCCCCGTTGTGTAGATAAC-3′ | (SEQ ID NO: 1) |

In the PCR, 50 ng of a constitutive high-level expression vector (PHCE DNA vector) as a template, 10 pmol of the primer of SEQ ID NO: 1, and 2 units of ExTaq DNA polymerase (TaKaRa, Japan), were added to 50 μl of a reagent composition containing 10 mM Tris HCl (pH 9.0), 1.5 mM magnesium chloride, 50 mM potassium chloride, 0.1% Triton X-100 and 150 μM of four kinds of deoxynucleotide triphosphates (DATP, dTTP, dGTP, dCTP), and then, 30 cycles of PCR amplification, each cycle consisting of changes of temperature, 30 seconds at 94° C., 30 seconds at 50° C. and 3 minutes at 72° C., were performed in a PCR reactor (iCycler, BIO-RAD, USA).

Thereafter, in order to introduce AspEI restriction enzyme recognition sites at the downstream of the promoter of pHCE-M1, PCR using primers of SEQ ID NOS: 2 and 3 containing an AspEI restriction enzyme recognition site (5′-GACNNN↓NNGTC-3′) was performed to produce pHCE-M2 where two AspEI restriction enzyme recognition sites were introduced at the downstream of the HCE promoter of the pHCE-M1. In the following primer base sequences of SEQ ID NOS: 2 and 3, the restriction enzyme recognition sites are underlined portions.

(SEQ ID NO: 2)

5′-TCCGACATATGGTCATCTCCTTCGGTATATCTCCTTTTTCCAG-3′

(SEQ ID NO: 3)

5′-GGACTTAAGGTCGGATCATTAGTTCCGCGTGGC-3′

In the PCR, the pHCE-M1 as a template, 10 pmol of the primers of SEQ ID NOS: 2 and 3, and 2 units of ExTaq DNA polymerase (TaKaRa, Japan) were added to the same reagent composition used as in the above PCR, and then 30 cycles of PCR amplification, each cycle consisting of changes of temperature, 30 seconds at 94° C., 30 seconds at 50° C. and 3 minute at 72° C., was performed.

In order to make DNA separation easy in converting the plasmid pHCE-M2 obtained as described above into a T-vector, a 800-bp DNA amplification product having AspEI restriction enzyme recognition sites at its both ends was inserted between the two AspEI restriction enzyme recognition sites, thereby producing a plasmid (pHCE-FOREX). This 800-bp DNA amplification product was obtained by PCR using the primers of SEQ ID NOs: 4 and 5 containing AspEI restriction enzyme recognition sites. In this PCR, the same reagent composition as used in the above PCR was used, and 30 cycles of PCR amplification, each cycle consisting of changes of temperature, 30 seconds at 94° C., 30 seconds at 50° C. and 1 minute at 72° C., was performed in a PCR reactor (iCycler, BIO-RAD, USA).

(SEQ ID NO: 4)

5′-GACCATATGTCGAAAGTTTATATTAGTGGAG-3′

(SEQ ID NO: 5)

5′-GACCTTAAGTCCAGTTAAAAACTGCAATATTCG-3′

The produced plasmid pHCE-FOREX for constitutive high-level expression T-vectors functions as both a T-vector and an expression vector.

### Example 2

### Conversion of Plasmid for T-Vector into T-Vector

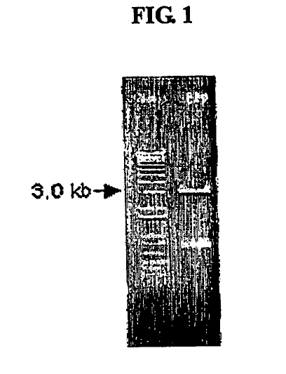

In order that the plasmid (pHCE-FOREX) for constitutive high-level T-vectors obtained in Example 1, into which the 800-bp DNA fragment containing the AspEI restriction enzyme recognition sites at its ends have been cloned, is converted into a T-vector, the plasmid for T-vectors, which have been separated and purified to high purity, was treated with an AspEI restriction enzyme (10 units per 3 μg DNA) at 37° C. for 6 hours, and then electrophoresed on 1% agarose gel (FIG. 1).

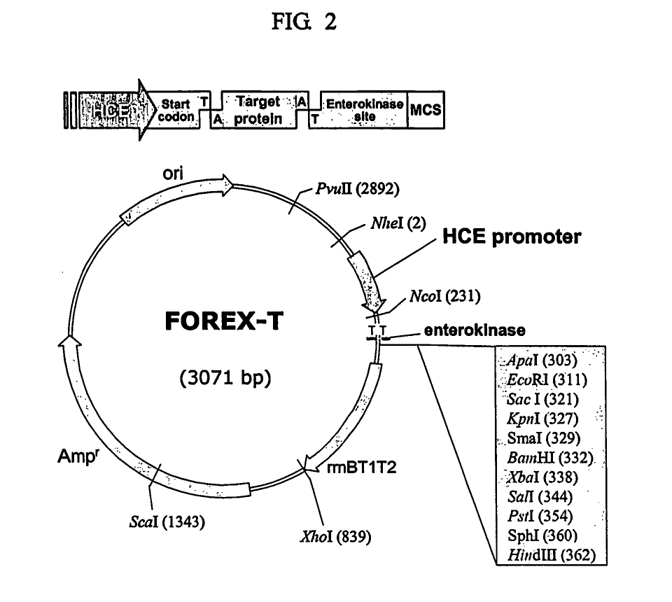

In FIG. 1, the first lane represents a 1-kb plus DNA ladder (Promega Co. USA), and the second lane represents the positions of a T-vector and gene resulted from the treatment (two-step digesting) of the plasmid (pHCE-FOREX) constitutive for high-level expression T-vectors with AspEI. As shown in FIG. 1, it could be found that when the plasmid pHCE-FOREX for T-vectors was cut two times with the AspEI restriction enzyme, the difference in the distance of migration on agarose gel between the cut polynucleotides was significant. This indicates that the DNA fragment for T-vectors can be easily separated. The T-vector DNA fragment of about 3,000 bp cut upon treatment with AspEI was purified with a gel purification kit (Bioneer, Korea), and then used as a T-vector (pHCE-FOREX-T). FIG. 2 is a schematic diagram showing the structure of pHCE-FOREX-T that is a new, constitutive high-level expression T-vector.

### Example 3

### Cloning Using T-Vector (PHCE-FOREX-T) Converted from pHCE-FOREX

In order to verify the cloning efficiency of the T-vector (pHCE-FOREX-T), which have been converted by treating the plasmid (pHCE-FOREX) for constitutive high-level expression vectors with restriction enzyme AspEI, a human tumor necrosis factor-α (hTNF-α) gene was amplified by PCR, and then cloned into the T-vector.

To amplify the hTNF-α gene, the primer of SEQ ID NO: 6 having ATG inserted into a fragment of the gene, and the base sequence-specific primer of SEQ ID NO: 7, were designed.

| | |
|---|---|
| 5′-ATGGTCAGATCATCTTCTC-3′ | (SEQ ID NO: 6) |
| 5′-CAGGGCAATGATCCAAAG-3′ | (SEQ ID NO: 7) |

Next, 10 pmol of the primers of SEQ ID NOS: 6 and 7, and 2 units of ExTaq DNA polymerase (TaKaRa, Japan), were added to 50 μl of a reagent composition containing 10 mM Tris HCl (pH 9.0), 1.5 mM magnesium chloride, 50 mM potassium chloride, 0.1% Triton X-100, and 150 μM of four kinds of deoxynucleotide triphosphates (DATP, dTTP, dGTP, and dCTP), and then, a 30-cycle PCR was performed, each cycle consisting of changes of temperature, at 94° C. for 30 seconds, at 52° C. for 30 seconds, and at 72° C. for 40 seconds, in a PCR reactor (iCycler, BIO-RAD, USA).

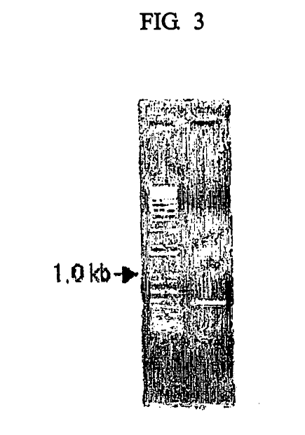

The gene amplification product was analyzed by electrophoresis on 1% agarose gel, and then purified with a gel purification kit (Bioneer, Korea) (FIG. 3). In FIG. 3, the first lane represents a 1-kb plus DNA ladder (Promega Co. USA), and the second lane represents the purified hTNF-α gene amplification product with the size of 472 bp. 50 ng of the T-vector prepared in Example 2, and the amplified and purified hTNF-α gene amplification product, were ligated with each other by 5 units of a T4 DNA ligase (TaKaRa, Japan), and introduced into E. coli JM109. The transformed E. coli was cultured in 5 ml LB medium, and then, the plasmid was separated and examined whether the hTNF-α was cloned into the plasmid.

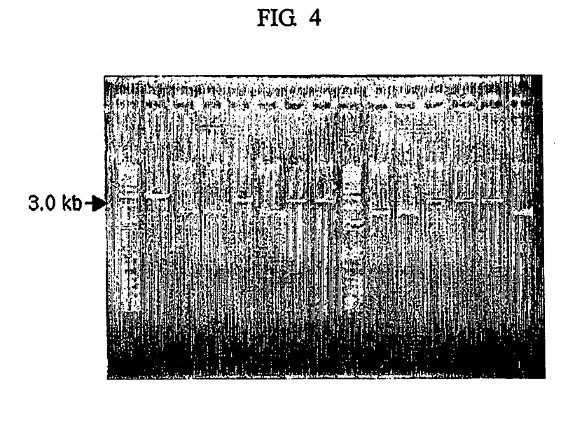

12 colonies that have been randomly selected from the obtained transformants were analyzed and the result showed that the hTNF-α was inserted into all the colonies. This indicates that the plasmid has high cloning efficiency. When cloning a gene amplification product with a primer having an ATG starting codon, if the gene is inserted into a HCE promoter in the forward direction, an NdeI restriction enzyme recognition site is produced. Using this fact, the cloning direction was examined by NdeI restriction enzyme digesting, and the result showed that 6 colonies out of the 12 colonies were cloned into the HCE promoter in the forward direction (FIG. 4).

In FIG. 4, the first and ninth lanes represent a 1-kb plus DNA ladder (Promega Co. USA), the second lane represents a control group for the comparison between DNA sizes, which is pHCE-FOREX cut with EcoRI having only one recognition site, and the third to eighth lanes and the tenth to fourteenth lanes, represent DNAs that were obtained from the 12 colonies, cut with a NdeI restriction enzyme and developed. In the case of six colonies developed on the fifth, seventh, eighth, twelfth, thirteenth, and fourteenth, a single DNA fragment of about 3.5 kb cut with NdeI was observed. This indicates that, in these six colonies, hTNF-α was cloned into T-vector in the forward direction.

### Example 4

### Confirmation of Protein Expressed from Cloned Gene Amplification Product

In the six colonies examined by the NdeI restriction enzyme treatment, a gene encoding hTNF-α was inserted into the HCE promoter in the forward reaction. Thus, using the characteristic as an expression vector of the inventive T-vector, the expression of hTNF-α can be examined directly without a re-cloning process or transformation into other host cells.

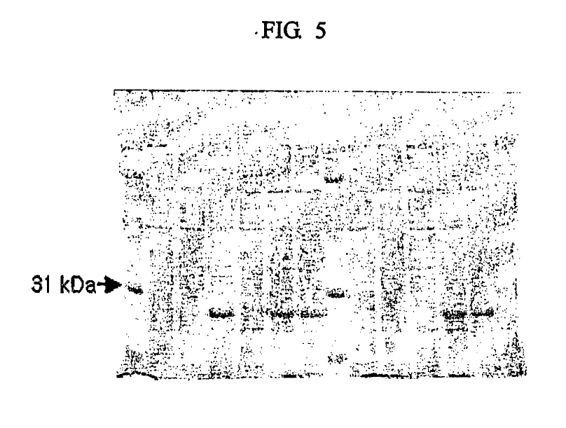

The 12 colonies found to have been cloned were cultured in LB medium for 20 hours, and then, each 10 μg of a protein obtained from each of the cultured cells was separated by 12% SDS-PAGE, stained with dye (Brilliant Blue R250) and examined whether hTNF-α was expressed or not (FIG. 5).

The results showed that the band of highly expressed hTNF-α was found in the fourth, sixth, seventh, twelfth and thirteenth lanes, and in 5 colonies out of the six colonies where the gene have been inserted into the HCE promoter in the forward direction, the high-level expression of the gene was successful. In FIG. 5, the first and eighth lanes represent a low molecular weight marker (Amersham, USA), and the second to seventh lanes and the ninth to fourteenth lanes represent 10 μg of the developed protein resulted from the culturing of the 12 transformants obtained as described above. The order of development of the test groups was the same as that in FIG. 4.

While the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described and proved in detail above, the inventive plasmid functioning as both the T-vector and the expression vector is easily converted into the T-vector, and allows target protein expression to be examined by one-step cloning. Particularly, the AspEI restriction enzyme recognition sites in the inventive plasmid are placed at intervals of about 800 bp to make the distinction between cut vectors easy upon restriction enzyme digesting, and the inventive T-vector is in the form of a plasmid and thus has an excellent storage property.

Furthermore, the inventive expression vector has a very efficient property that allows target protein expression to be examined only by one-step cloning without a need for re-subcloning, so that it can be widely used in the cloning of a gene encoding a target protein to be expressed. Particularly, according to the present invention, expression plasmids for large amounts of target proteins can be produced at the same time, the present invention can be applied to the short-term establishment of expression systems for certain microbial genomes and gene groups. Moreover, since the inventive vector is the expression vector of a constitutive high-level expression system, it does not require treatment with an expression inducer, has a very high usefulness as a vector bound to an expression system, which does not require specific host cells.

1. A plasmid wherein two restriction enzyme recognition sites into which a T-vector can be cloned are introduced at the downstream of a promoter of a vector that is constantly expressed at high levels regardless of the kind of a host cell, whereby the plasmid functions as both the T-vector and an expression vector and has the property of allowing the expression of a target gene to be examined only by one-step T-vector cloning.

2. The plasmid according to claim 1, wherein the restriction enzyme recognition sites into which the T-vector can be cloned are selected from the group consisting of HphI, MboII, AspEI and XcmI, and a polynucleotide is inserted between the two restriction enzyme recognition sites.

3. The plasmid according to claim 2, wherein a nucleotide having thymine bases at both 3′-ends of the removal position of the inserted polynucleotide is exposed, when the plasmid is cut with the restriction enzymes.

4. The plasmid according to claim 1, wherein the constitutive high-level expression vector is pHCE.

5. A plasmid (pHCE-FOREX) functioning as both a T-vector and an expression vector, wherein two AspEI restriction enzyme recognition sites are introduced at the downstream of the HCE promoter of the pHCE vector, and a polynucleotide having AspEI restriction enzyme recognition sites at its both ends is inserted between the two AspEI restriction enzyme recognition sites.

6. A constitutive high-level expression T-vector (pHCE-FOREX-T), which is obtained by digesting the plasmid pHCE-FOREX of claim 5 with an AspEI restriction enzyme, to remove the polynucleotide having AspEI restriction enzyme recognition sites at its both ends, and in which a nucleotide having thymine bases at both 3′-ends of the removal position of the polynucleotide is exposed.

7. A method for producing a plasmid (pHCE-FOREX) functioning as both a T-vector and an expression vector, the method comprising the steps of (a) constructing pHCE-M1 which the restriction enzyme recognition sites were removed by inducing point mutation in AspEI restriction enzyme recognition sites in a pHCE vector; (b) constructing pHCE-M2 by introducing two AspEI restriction enzyme recognition sites into the downstream of the HCE promoter of the pHCE-M1 vector by PCR using primers containing the AspEI restriction enzyme recognition sites; and (c) inserting a polynucleotide having AspEI restriction enzyme recognition sites at its both ends, between the two AspEI restriction enzyme recognition sites of the pHCE-M2 vector.

8. An expression vector, which is obtained by digesting the plasmid of claim 2 with the restriction enzymes to remove the inserted polynucleotide, and then inserting a gene encoding a target protein, into a position from which the polynucleotide was removed.

9. An expression vector, wherein a gene encoding a target protein is inserted into the constitutive high-level expression T-vector (pHCE-FOREX-T) of claim 6.

10. The expression vector according to claim 8, wherein the target protein-encoding gene is a gene amplified by PCR.

11. The expression vector according to claim 8, wherein the target protein-encoding gene is a PCR product amplified by using a primer having the amino terminal end of ATG, and a primer specific to the base sequence of the gene.

12. The expression vector according to claim 8, wherein NdeI restriction enzyme recognition site is formed in the insertion position of the gene encoding the target protein.

13. A microorganism transformed with the expression vector of claim 8.

14. A method for expressing a gene encoding target protein, which comprises culturing the transformed microorganism of claim 13.

15. An expression vector library, which is prepared by the method comprising the steps of: (a) removing the inserted polynucleotide by digesting the plasmid of claim 2 with the restriction enzyme selected from the group consisting of HphI, MboII, AspEI and Xc7nI; and (b) inserting the library of various genes into a position from which the polynucleotide was removed.

16. An expression vector library wherein the library of various genes is inserted into the high-level expression T-vector (pHCE-FOREX-T) of claim 6.

17. A method for determining the cloning of a target gene, the method comprising the steps of: (a) transforming microorganisms with the expression vector library of claim 15; and (b) culturing the transformed microorganisms.

18. The method for determining the cloning of a target gene according to claim 17, further comprising the steps of: separating a plasmid after the step (b); and digesting the plasmid with an NdeI restriction enzyme.

19. The expression vector according to claim 9, wherein the target protein-encoding gene is a gene amplified by PCR.

20. The expression vector according to claim 9, wherein the target protein-encoding gene is a PCR product amplified by using a primer having the amino terminal end of ATG, and a primer specific to the base sequence of the gene.

21. The expression vector according to claim 9, wherein NdeI restriction enzyme recognition site is formed in the insertion position of the gene encoding the target protein.

22. A microorganism transformed with the expression vector of claim 9.

23. A microorganism transformed with the expression vector of claim 10.

24. A microorganism transformed with the expression vector of claim 11.

25. A microorganism transformed with the expression vector of claim 12.


SEQUENCE LISTING (7 sequences)

SEQ ID NO: 1 - DNA, 26 bp, Artificial
  PCR primer for deleting AspE1 site in pCHE vector
gcctggctccccgttgtgtagataac

SEQ ID NO: 2 - DNA, 43 bp, Artificial
  PCR primer containg AspEI site
tccgacatatggtcatctccttcggtatatctcctttttccag

SEQ ID NO: 3 - DNA, 33 bp, Artificial
  PCR primer containing AspEI site
ggacttaaggtcggatcattagttccgcgtggc

SEQ ID NO: 4 - DNA, 31 bp, Artificial
  PCR primer for 800 bp polynucleotide
gaccatatgtcgaaagtttatattagtgcag

SEQ ID NO: 5 - DNA, 33 bp, Artificial
  PCR primer for 800bp polynucleotide
gaccttaagtccagttaaaaactgcaatattcg

SEQ ID NO: 6 - DNA, 19 bp, Artificial
  PCR primer containg ATG sequence
atggtcagatcatcttctc

SEQ ID NO: 7 - DNA, 18 bp, Artificial
  PCR primer containg ATG sequence
cagggcaatgatccaaag